(19) United States
(12) Patent Application Publication
(10) Pub. No.: US 2003/0087917 A1
(43) Pub. Date: May 8, 2003

(54) Use of dopamine-d3 receptor ligands for the treatment of diseases of the central nervous system

(76) Inventors: Dorothea Strack, Ludwigshafen (DE); Hans-Jorg Treiber, Bruhl (DE); Liliane Unger, Ludwigshafen (DE); Hans-Jurgen Teschendorf, Dudenhofen (DE); Gerhard Gross, Speyer (GB)

Correspondence Address: KEIL & WEINKAUF, 1350 CONNECTICUT AVENUE, N.W., WASHINGTON, DC, 20036, US

(21) Appl. No.: 10/239,828

(22) PCT Filed: Mar. 26, 2001
(86) PCT No.: PCT/EP01/03411

(30) Foreign Application Priority Data
Mar. 27, 2000 (DE) 100 15 211.2

(51) Int. Cl. A61K 31/52; A61K 31/44; A61K 31/4439; A61K 31/4436; A61K 31/443; A61K 31/381; A61K 31/343

(52) U.S. Cl. 514/263.1; 514/263.2; 514/332; 514/336; 514/337; 514/443; 514/469

(57) ABSTRACT

The present invention relates to the use of at least one compound of the general formula I,

L-D-B-G (I)

in which L and G are aromatic, optionally heterocyclic radicals, D is an aliphatic or heteroaliphatic connecting link and B is a 6-, 7- or 8-membered saturated or unsaturated ring bonded to D via the 1 position and to G via the 4 or 5 position and having one or two nitrogen heteroatoms, for the treatment of disorders of the central nervous system and in particular of disorders which are to be assigned to the psychiatric or neurological type. These compounds offer particular advantages in the control of addiction.

### DESCRIPTION

The present invention relates to the use of certain dopamine D₃ receptor ligands for the treatment of disorders of the central nervous system, especially the treatment of psychological disorders which are mediated by psychotropic substances.

Neurons obtain their information, inter alia, by means of G protein-coupled receptors. There are numerous substances which exert their action via these receptors. One of these is dopamine.

Confirmed knowledge about the presence of dopamine and its physiological function as a neurotransmitter is available. Disturbances in the dopaminergic transmitter system result in disorders, such as schizophrenia, depression and Parkinson's disease. These and other disorders are treated with medicaments which interact with the dopamine receptors.

Until 1990, two subtypes of dopamine receptors were clearly defined pharmacologically, namely the D₁ and D₂ receptors.

More recently, a third subtype has been found, namely the D₃ receptor, which appears to mediate some effects of the antipsychotics and antiparkinson agents (J. C. Schwartz et al., The dopamine D₃ Receptor as a Target for Antipsychotics, in Novel Antipsychotic Drugs, H. Y. Meltzer, Ed. Raven Press, New York 1992, pages 135-144; M. Dooley et al., Drugs and Aging 1998, 12, 495-514).

At present, the dopamine receptors are divided into two families, on the one hand the D₂ group consisting of D₂, D₃ and D₄ receptors, on the other hand the D₁ group consisting of D₁ and D₅ receptors. While D₁ and D₂ receptors are widespread, D₃ receptors, however, appear to be expressed regioselectively. Thus these receptors are preferably found in the limbic system, the projection areas of the mesolimbic dopamine system, especially in the nucleus accumbens, but also in other areas, such as the amygdala. Because of this comparatively regioselective expression, D₃ receptors count as a low-side effect target, and it is assumed that a selective D₃ ligand should presumably have the properties of known antipsychotics, but not their dopamine D₂ receptor-mediated neurological side effects (P. Sokoloff et al., Localization and Function of the D₃ Dopamine Receptor, Arzneim. Forsch./Drug Res. 42(1), 224 (1992); P. Sokoloff et al. Molecular Cloning and Characterization of a Novel Dopamine Receptor (D₃) as a Target for Neuroleptics, Nature, 347, 146 (1990)).

Selective dopamine D₃ receptor ligands are known. For example, a substance of the formula

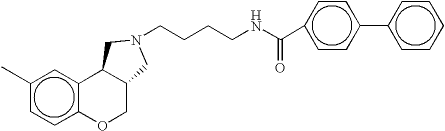

is mentioned in Dubuffet et al. (1999) Bioorg. Med. Chem. Lett. 9, 2059-2064, or a substance of the formula

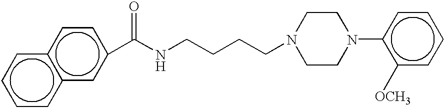

is mentioned in EP 779 284 and Pilla et al. (1999) Nature 400, 371-375. Further arylpiperazines having selective affinity for dopamine D₃ receptors are described by P. J. Murray et al. in Bioorganic & Medicinal Chemistry Letters, Vol. 5, No. 3, 219-222 (1995).

Further compounds for the treatment of disorders which respond to dopamine D₃ ligands are disclosed in WO 96/02519; WO 96/02520; WO 96/02249; WO 96/02246; WO 97/25324; WO 99/02503 and WO 98/05178. As is known, these compounds can be used for the treatment of schizophrenia, depression, neuroses and psychoses.

A considerable need still exists for effective treatment possibilities of various disorders of the central nervous system. For example, addiction phenomena and related disorders of the psyche and of the behavior of drug- or pharmaceutical-dependent individuals in many cases can only be inadequately controlled at present. While heroin dependence can be treated with opioid agonists, e.g. methadone, there is still no pharmacotherapy against cocaine abuse at present.

Surprisingly, it has now been found that certain compounds have a suitable pharmacological profile of action such that they can be employed for the treatment of various disorders of the central nervous system.

The subject of the present invention is therefore the use of at least one compound of the general formula I

L-D-B-G (I)

in which

L is a 5- or 6-membered aromatic heteromonocyclic system L1 having 1, 2 or 3 heteroatoms selected independently of one another from O, N and S

or an aromatic or heteroaromatic radical selected from the group L2

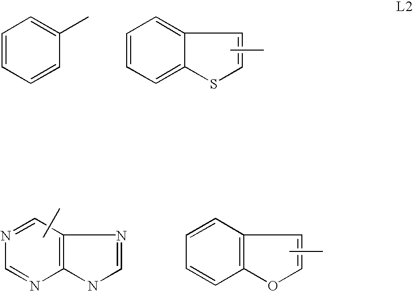

where L optionally has 1, 2, 3 or 4 substituents which independently of one another are selected from C₁-C₈-alkyl which is optionally substituted by OH, OC₁-C₈-alkyl, phenyl or halogen; OR¹, C₂-C₆-alkenyl, C₂-C₆-alkynyl, C₃-C₈-cycloalkyl, halogen, CN, CONR¹R², COOR¹, NO₂, NR¹R², SR¹, SO₂R¹, SO₂NR¹R², OSO₂R¹, Ax1 or phenoxy which is optionally substituted by C₁-C₆-alkyl, OC₁-C₆-alkyl or halogen; C₁-C₆-alkanoyl or benzoyl;

in which

Ax1 is phenyl, naphthyl or a 5- or 6-membered heterocyclic aromatic ring having 1, 2, 3 or 4 heteroatoms,

where A×1 optionally has 1, 2, 3 or 4 substituents which independently of one another are selected from C₁-C₆-alkyl which is optionally substituted by OH, OC₁-C₆-alkyl, halogen or phenyl; C₁-C₆-alkoxy, C₂-C₆-alkenyl, C₂-C₆-alkynyl, C₃-C₆-cycloalkyl, halogen, CN, COR¹, COOR¹, NR¹R², NO₂, SR¹, SO₂R¹, SO₂NR¹R², or phenyl which is optionally substituted by C₁-C₆-alkyl, OC₁-C₆-alkyl, NR¹R², CN, CF₃, CHF₂, or halogen; and where the heterocyclic aromatic ring mentioned can optionally be fused to a phenyl ring;

R¹ is H, C₃-C₆-cycloalkyl, phenyl or C₁-C₈-alkyl which is optionally substituted by OH, OC₁-C₈-alkyl, halogen or phenyl;

R² has the meanings indicated for R¹ or is COR¹ or CO₂R¹;

D is a C₁-C₁₈-alkylene group or a C₁-C₁₈-alkylene group which includes at least one group Z which is selected from O, S, NR², C₃-C₆-cycloalkyl, CO, CONR², CH₂, a double and a triple bond, where R² is as defined above;

B is a 6-, 7- or 8-membered saturated ring having one or two nitrogen heteroatoms, where the nitrogen heteroatoms are located in the 1,4 or 1,5 position and the ring is bonded to the radical D in the 1 position and to the radical G in the 4 or 5 position and where the ring can moreover have a double bond in the 3 or 4 position;

G is phenyl, pyridyl, pyrimidinyl or triazinyl,

where G can optionally have 1, 2, 3 or 4 substituents which independently of one another are selected from OR¹, C₁-C₈-alkyl, C₂-C₈-alkenyl, C₂-C₈-alkynyl, C₁-C₆-alkoxy-C₁-C₆-alkyl, halogen-C₁-C₆-alkyl, halogen-C₁-C₆-alkoxy, halogen, CN, CO₂R¹, NO₂ SO₂R¹, SO₃R¹, NR¹R², SO₂NR¹R², SR¹, a 5- or 6-membered carbocyclic, aromatic or nonaromatic ring and a 5- or 6-membered heterocyclic aromatic or nonaromatic ring having 1 or 2 heteroatoms independently of one another selected from O, S and N, where the carbocyclic or the heterocyclic ring is optionally substituted by C₁-C₈-alkyl, phenyl, phenoxy, halogen, OC₁-C₈-alkyl, OH, NO₂, or CF₃,

where G can optionally be fused to a carbocyclic or heterocyclic ring of the type defined above;

and their salts with physiologically tolerable acids,

for the production of a medicament for treating disorders of the central nervous system which respond to dopamine D₃ ligands.

Disorders of the central nervous system are understood as meaning disorders which concern the spinal chord and especially the brain. The term “disorder” in the sense according to the invention describes anomalies which as a rule are regarded as pathological conditions or functions and can be shown in the form of certain signs, symptoms and/or malfunctions. The treatment according to the invention can be directed at individual disorders/anomalies or pathological conditions; however, a number of anomalies which are optionally causally connected to one another can be combined to give models, i.e. syndromes, which can be treated according to the invention.

The disorders which can be treated according to the invention especially include psychiatric and neurological disorders. These in particular include organic disorders, including symptomatic disorders, such as psychoses of the acute exogenous reaction type or concomitant psychoses of organic or exogenous cause, e.g. after trauma, especially brain lesions and diffuse brain damage, in metabolic disorders, infections and endocrinopathies; endogenous psychoses, such as schizophrenia and schizotypic and delusional disorders; affective disorders, such as depressions, mania or manic depressive conditions; and mixed forms of the disorders described beforehand; neurotic and somatoform disorders and disorders associated with stress; dissociative disorders, e.g. lapses, clouding and dissociation of consciousness and personality disorders; disorders of attention and waking/sleeping behavior, such as behavioral disorders and emotional disorders, which begin in childhood and adolescence, e.g. hyperactivity in children, intellectual deficits, in particular attention deficit disorders, memory and cognitive disorders, e.g. learning and memory impairment (impaired cognitive function), dementia, narcolepsy and sleep disorders, e.g. restless legs syndrome; development disorders; anxiety states; delirium; disorders of the sex life, e.g. male impotence; eating disorders, e.g. anorexia and bulimia; addiction; and further psychiatric disorders not defined in detail.

The disorders which can be treated according to the invention also include neurodegenerative disorders, i.e. in particular disorders as a result of neuronal damage. These include the neuronal damage in particular accompanying parkinsonism; epilepsy and ischemic events, especially stroke, and in particular the affective disorders associated therewith.

Preferred embodiments of the present invention lie in the treatment of the psychoses and affective disorders mentioned beforehand, the treatment of addiction or the treatment of disorders of the attention and of the waking/sleeping behavior.

The term “addiction” according to the invention stands for the dependence of an individual on exogenous and/or endogenous stimuli and/or habituation to exogenous and/or endogenous stimuli.

The dependence can be of a physical and/or psychological nature.

Physical dependence can be manifested, in particular, in a withdrawal syndrome. The withdrawal syndrome is an undesired physiological change which occurs, for example, when the intensity of an addiction-mediated stimulus is decreased, or the stimulus is counteracted and in particular the stimulus is suppressed.

Physical dependence can be accompanied by a feeling of satisfaction and the desire to repeat the stimulus.

Habituation as a feature characterizing addiction describes the circumstance of having to increase the intensity of a stimulus progressively in order to be able to achieve a specific action.

According to a further aspect of the present invention, the term “addiction” stands for disorders of the psyche and of the behavior of an individual which are associated with addiction-mediating stimuli. These especially include a behavior typical of addiction, in particular a compulsive behavior or intense craving and/or the predominant arrangement of individual activities in order to supply addiction-mediating stimuli.

According to the invention, a differentiation is made between addiction which is caused by exogenous factors, and addiction as a result of endogenous factors.

Exogenous factors especially include psychotropic substances. A psychotropic substance in the sense according to the invention can be one which brings about habituation, physical dependence and/or psychological dependence in an organism. Dependence in connection with the use of psychotropic substances can also be described by the term drug dependence.

Substances bringing about dependence in particular include those which act on the central nervous system. Actions in this sense are especially a lowering of anxiety and tension, changes in the state of mind which are perceived as pleasant by the affected person, e.g. elated mood or euphoria, the sensation of an increased mental and/or physical capacity, a modified sensory awareness and/or behavioral changes.

A particular group of addictive disorders to be treated according to the invention are those wherein there is a pattern of habituation and psychological dependence and in particular cases also physical dependence.

Endogenous factors in particular include disorders of transmitter systems, especially of the dopaminergic system. Thus compulsive gambling, for example, can be treated according to the invention.

Psychotropic substances are described in other connections as toxins, in particular luxury food, drink and tobacco toxins, pharmaceuticals, drugs or solvents. These substances include, for example, stimulants, such as opioids, e.g. morphine, heroin and codeine; amphetamine-like substances, e.g. amphetamine, methylphenidate and cocaine; nicotine; alcohol; substances which interact with the GABA chloride channel complex; sedatives; anxiolytics, hypnotics or tranquilizers, e.g. benzodiazepines and barbiturates; hallucinogenics, e.g. LSD; cannabinoids, e.g. marijuana; psychomotor stimulants, such as 3,4-methylenedioxy-N-methylamphetamine (ecstasy); phencyclidine; or other stimulants, including caffeine. In the context of the treatment according to the invention, disorders which are especially to be taken into consideration are those which are mediated by opioids, in particular codeine, by amphetamine-like substances, in particular cocaine or amphetamine, nicotine and alcohol.

A particular embodiment of the present invention aiming at addiction treatment especially relates to the treatment of addiction symptoms, such as withdrawal symptoms, compulsive behavior and intensive craving with respect to the addictive substance(s). The aim of the treatment according to the invention is in particular to decrease and preferably to suppress the expression and strength of the addiction symptoms perceived by the affected individual such that, according to a particular aspect of the present invention, weaning from the addiction is facilitated and/or the relapse frequency into addiction is decreased after abstinence. The present invention thus relates to a treatment directed in particular at the taking and, under certain circumstances, the abuse of psychotropic substances, the motivation for addiction especially being decreased.

According to a further aspect of the present invention, the treatment is directed at disorders whose causes are at least partially to be attributed to an anomalous activity of dopamine D₃ receptors.

According to another aspect of the present invention, the treatment is especially directed at those disorders which can be influenced by binding of preferably exogenously added binding components (ligands) to dopamine D₃ receptors in the sense of an expedient medicinal treatment. According to a particular embodiment, those disorders are treated which can be influenced by an at least partial activation of dopamine D₃ receptors. This includes a partial and also a complete agonist action of dopamine D₃ receptors.

Diseases to be treated according to the invention are often characterized by progressive development, i.e. the conditions described above change in the course of time, as a rule the degree of severity increases and conditions can optionally change into one another or conditions further to already existing conditions can occur.

By means of the treatment according to the invention of disorders of the central nervous system, a large number of signs, symptoms and/or malfunctions can be treated which are associated with the disorders and in particular the abovementioned conditions. These include, for example, a disturbed regard for reality, lack of sense and ability to meet customary social standards and/or living demands, character changes, changes in individual drive, such as hunger, sleep, thirst, etc., and emotional state, disturbances of the memory and capacity of association, personality changes, in particular affective instability, hallucinations, ego disorders, distractedness, ambivalence, autism, depersonalization and/or hallucinations, delusions, syllabication, absent synkinesis, small-step gait, bent posture of trunk and limbs, tremor, parkinsonian mask, monotonous speech, depression, apathy, impeded spontaneity and resoluteness, poor association ability, anxiety, nervous unrest, stuttering, social phobia, panic disorders, withdrawal syndromes in the case of dependence, expansive syndromes, states of excitation and confusion, dysphoria, dyskinetic syndromes and tick disorders, e.g. Huntington's chorea, Gilles de la Tourette syndrome, vertigo syndromes, e.g. peripheral positional, rotary and vestibular vertigo, melancholy, hysteria, hypochondria and the like.

A treatment in the sense according to the invention comprises not only the treatment of acute or chronic signs, symptoms and/or malfunctions but also a preventive treatment (prophylaxis), in particular as a recurrence or phase prophylaxis. The treatment can be accomplished symptomatically, for example as symptom suppression. It can be carried out short-term, be accomplished medium-term, or it can also be a long-term treatment, for example in the course of a maintenance therapy.

According to the invention, at least one compound of the general formula I having the meanings mentioned at the outset is used for the treatment of the abovementioned indications. If the compounds of the formula I have one or more centers of asymmetry, enantiomer mixtures, in particular racemates, diastereomer mixtures, tautomer mixtures, but preferably the respective essentially pure enantiomers, diastereomers and tautomers, can also be employed.

Likewise utilizable are physiologically tolerable salts of the compounds of the formula I, especially acid addition salts with physiologically tolerable acids. Suitable physiologically tolerable organic and inorganic acids are, for example, hydrochloric acid, hydrobromic acid, phosphoric acid, sulfuric acid, oxalic acid, maleic acid, fumaric acid, lactic acid, tartaric acid, adipic acid or benzoic acid. Further utilizable acids are described in Fortschritte der Arzneimittelforschung, Volume 10, pages 224 ff., Birkhäuser Verlag, Basle and Stuttgart, 1966.

Terms such as alkyl, alkoxy, etc. include straight-chain or branched hydrocarbon groups, such as CH₃, C₂H₅, n-propyl, CH(CH₃)₂, n-butyl, CH(CH₃)C₂H₃, 2-methylpropyl, C(CH₃)₃, n-pentyl or n-hexyl, in particular CH₃, C₂H₅, CH(CH₃)₂ or C(CH₃)₃, preferably having—if not stated otherwise—1 to 8, in particular 1 to 6 and particularly preferably 1 to 4, carbon atoms.

Substituted “alkyl, alkoxy, etc.” in particular include:

Haloalkyl, i.e. alkyl which is partially or completely substituted, in particular substituted 1, 2, 3 or 4 times, by identical or different halogen atoms, preferably in the α or ω position, that is, for example, CH₂F, CHF₂, CF₃, CH₂Cl, CF₂Cl, 2-fluoroethyl, 2-chloroethyl or 2,2,2-trifluoroethyl, where CF₃, CHF₂, CF₃Cl and CH₂F are particularly preferred;

Haloalkoxy, i.e. alkoxy which is partially or completely substituted, in particular substituted 1, 2, 3 or 4 times by identical or different halogen atoms, preferably in the α or ω position, that is, for example, the haloalkoxy radicals corresponding to the haloalkyl radicals listed above;

Alkoxyalkyl, i.e. alkyl substituted by alkoxy, that is, for example, CH₂OCH₃ or 2-methoxyethyl;

Hydroxyalkyl, i.e. alkyl which is preferably monosubstituted by hydroxyl, e.g. hydroxymethyl or 2-hydroxyethyl;

Phenylalkyl, i.e. alkyl which is preferably monosubstituted by phenyl, e.g. benzyl or phenylethyl.

The term “cycloalkyl” includes mono- or bicyclic saturated hydrocarbon groups, such as cyclopropyl, cyclobutyl, cyclopentyl, cyclohexyl, etc., preferably having—if not stated otherwise—3 to 9, in particular 3 to 6 and particularly preferably 5 or 6, carbon atoms.

The term “alkenyl” includes straight-chain or branched unsaturated hydrocarbon groups which preferably have a double bond, such as ethenyl, prop-2-en-1-yl, etc., preferably having—if not stated otherwise—2 to 8, in particular 2 to 6 and particularly preferably 2 to 4, carbon atoms.

The term “alkynyl” includes straight-chain or branched unsaturated alkyl groups which preferably have a triple bond, such as ethynyl, prop-2-in-1-yl, etc., preferably having—if not stated otherwise—2 to 8, in particular 2 to 6 and particularly preferably 2 to 4, carbon atoms.

Alkanoyl means CO-alkyl, e.g. acetyl.

The term “alkylene” includes straight-chain or branched radicals, such as methylene, eth-1,1-ylene, eth-1,2-ylene, prop-1,1-ylene, prop-1,2-ylene, prop-1,3-ylene, prop-2,2-ylene, but-1,1-ylene, but-1,2-ylene, but-1,3-ylene, but-1,4-ylene, but-2,2-ylene, 2-methylprop-1,3-ylene, pent-1,1-ylene, pent-1,2-ylene, pent-1,3-ylene, pent-1,4-ylene, pent-1,5-ylene, pent-2,2-ylene, pent-2,3-ylene, pent-2,4-ylene, pent-3,3-ylene, 1-methylbut-1,4-ylene, 2-methylbut-1,4-ylene, etc., preferably having—if not stated otherwise—1 to 18, in particular 3 to 10 and particularly preferably 3 to 8, carbon atoms. In connection with the group D, these radicals can include one or more radicals, such that alkylene radicals can result radicals whose carbon chain is interrupted by one or more radicals Z or in which saturated bonds are replaced by unsaturated bonds (alkenylene; alkynylene). Thus straight-chain or branched unsaturated radicals can result, the number and arrangement of the carbon atoms of which corresponds to those of the previously mentioned alkylene radicals, but where one or more single bonds are replaced by corresponding unsaturated double or triple bonds.

The term “halogen” includes a fluorine, chlorine, bromine or iodine atom and in particular a fluorine or chlorine atom.

The term “heterocyclic radical” in particular includes 5- and 6-membered heterocyclic rings, which can be aromatic or nonaromatic, mono- or bicyclic, and/or benzofused, preferably having—if not stated otherwise—1, 2, 3 or 4, identical or different, heteroatoms selected from O, S and N. These especially include pyridinyl, pyrimidinyl, pyrazinyl, imidazolyl, indolyl, benzofuranyl, benzothienyl, pyrrolyl, furanyl, pyrazolyl, thienyl, oxazolyl, thiazolyl, isoxazolyl, tetrazolyl, triazinyl, thiadiazolyl and triazolyl.

The compounds which can be used according to the invention are composed of four structural elements L, D, B and G in the arrangement given according to formula I. Two of these groups, namely L and G, are aromatic carbocyclic or heterocyclic ring systems which optionally can also be substituted. As a rule, the group D as a binding member between L and B is an aliphatic or heteroaliphatic radical having a chain length of preferably at least four atoms. The linkage to the radical L advantageously takes place via a functionality. In particular, amide, ester, ether and preferably thioether bonds are formed there. At the same time, the carbonyl groups of amide or ester bonds are preferably on the aromatic system. The group B as a binding member between D and G is a heteroalicyclic radical which, as a rule, is bonded to D via a nitrogen atom. The linkage to G is variable within certain limits, but it should lead to an acceptable distance from D and G, which is why the linkage points are advantageously selected in the 1,4 or 1,5 position.

L is preferably one of the following radicals:

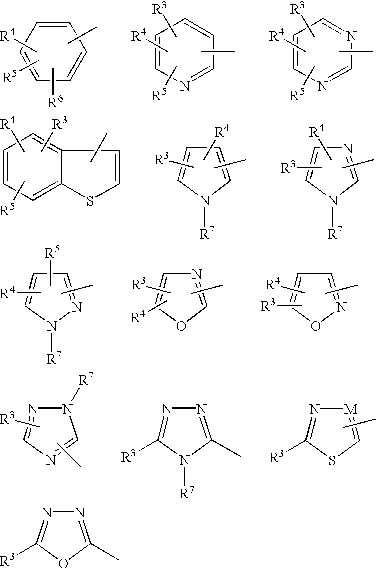

in which

R³ is Ax1, OR¹, R¹, C₂-C₆-alkenyl, C₂-C₆-alkynyl, halogen, CN, CONR¹R², COOR¹, NO₂, NR¹R², SR¹, OSO₂R¹, SO₂R¹;

R⁴ to R⁶ independently of one another are H, C₁-C₆-alkyl, OR¹, CN, NR¹R², SR¹, CF₃;

R⁷ is H, C₁-C₆-alkyl or C₃-C₆-cycloalkyl;

M is N or CR¹.

Particularly preferably, L is one of the following radicals:

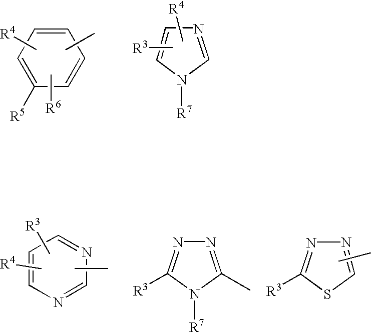

in which

R³ is Ax1, R¹, COOR¹, NO₂, NR¹R², SR¹, OSO₂CF₃, SO₂R¹, CF₃, CHF₂,

R⁴ to R⁶ are H, C₁-C₆-alkyl, OR¹, NR²R²;

R⁷ is H, C₁-C₆-alkyl.

Ax1 is preferably one of the following substituents:

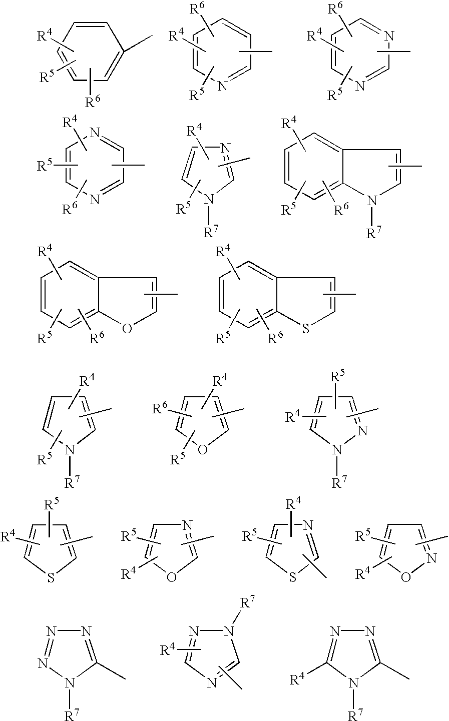

in which

R⁴ to R⁶ have the meanings indicated above;

R⁷ is preferably C₁-C₄-alkyl.

Particularly preferably, Ax1 is one of the following substituents:

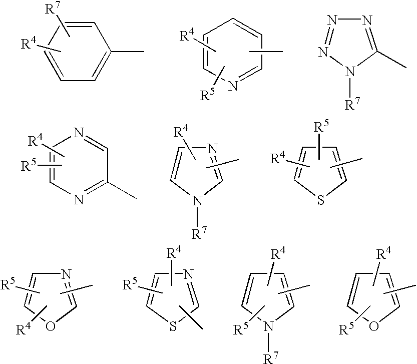

in which R⁴ to R⁶ have the meanings indicated above and R⁷ is preferably C₁-C₄-alkyl.

The phenyl, pyrazinyl, pyridyl and pyrrolyl substituents indicated above are particularly preferred.

Ax1 preferably substitutes triazolyl, in particular triazol-5-yl in the 3 position.

D is preferably a C₄-C₁₀-alkylene group or a C₃-C₁₀-alkylene group including Z, where the group Z is selected from O, S, NR², C₃-C₆-cycloalkyl, CO, CONR², CH₂, a double bond and a triple bond and one or more identical or different groups Z can be present in the alkylene group.

If the alkylene groups include at least one of the groups Z, these can be arranged in the alkylene chain in any desired position and in particular in position 1 or 2 of the group D (seen from the radical L). The radicals CONR² and COO are preferably arranged such that in each case the carbonyl group is facing toward the radical L. Particularly preferably, D is -Z-C₃-C₆-alkylene or -Z-C₃-C₆-alkenylene, in particular -Z-CH₂CH₂CH₂—, -Z-CH₂CH₂CH₂CH₂—, -Z-CH₂CH═CHCH₂—, -Z-CH₂C(CH₃)═CHCH₂—, -Z-CH₂C(═CH₂)CH₂—, -Z-CH₂CH(CH₃)CH₂— or is a linear -Z-C₇-C₁₀-alkylene radical, where Z is bonded to the radical L. Z is preferably CH₂, O and in particular S. Additionally preferably, D is —C₄-C₁₀-alkylene or —C₄-C₁₀-alkenylene, in particular —(CH₂)₄—, —(CH₂)₅—, —(CH₂)₆—, —CH₂CH₂CH═CHCH₂—, —CH₂CH₂C(CH₃)═CHCH₂—, —CH₂C(═CH₂)CH₂— or —CH₂CH₂CH(CH₃)CH₂—.

B is preferably one of the following radicals:

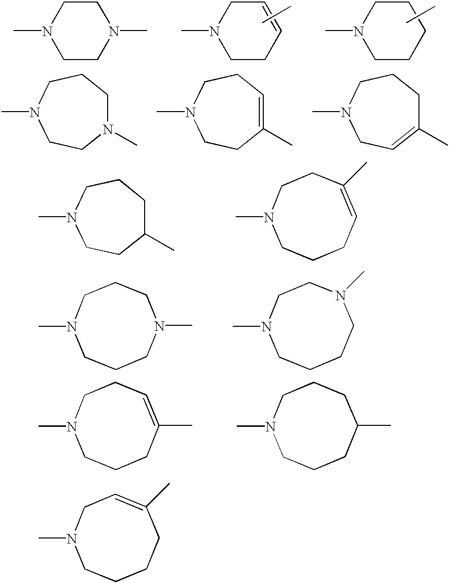

of these radicals, the following radicals are particularly preferred:

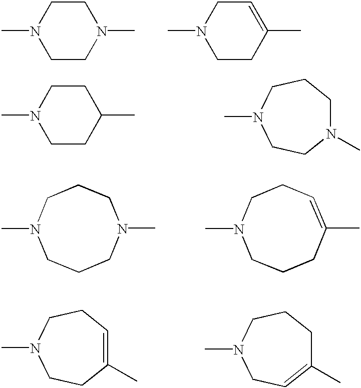

which are designated by B1 below.

The following radicals are particularly preferred:

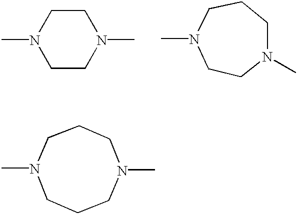

which are designated by B2 below.

The group G can have one, two, three or four substituents, preferably one or two substituents, which can be, in particular, in the m position and/or p position. These substituents are preferably selected from C₁-C₆-alkyl, halogen-C₁-C₆-alkyl, NO₂, CN, CO₂R¹, halogen, in particular chlorine, phenyl, pyrrolyl, imidaazolyl, pyrazolyl, thienyl, cyclopentyl and cyclohexyl.

If one of the substituents is C₁-C₆-alkyl, a branched group and in particular isopropyl or t-butyl is preferred.

Preferably, G is optionally substituted phenyl, 2-, 3- or 4-pyridinyl or 2-, 4(6)- or 5- pyrimidinyl.

If one of the substituents of the group G is a 5- or 6-membered heterocyclic ring, it is, for example, a pyrrolidine, piperidine, morpholine, pyridine, pyrimidine, triazine, pyrrole, thiophene or pyrazole radical, where a pyrrole, pyrrolidine, pyrazole or thienyl radical is preferred.

If one of the substituents of the radical G is a carbocyclic radical, it is in particular a phenyl, cyclopentyl or cyclohexyl radical.

If G is fused to a carbocyclic radical, it is in particular a naphthalene, di- or tetrahydronaphthalene radical.

In substituents OR¹, R¹ is preferably H, alkyl, CF₃, CHF₂ or phenyl. Particularly preferably, OR¹ is methoxy, trifluoromethoxy or phenoxy.

In substituents COOR¹, R¹ is H or alkyl. Particularly preferably, COOR¹ is alkoxycarbonyl, such as methoxycarbonyl, ethoxycarbonyl, n-propoxycarbonyl, i-propoxycarbonyl, n-butoxycarbonyl or t-butoxycarbonyl.

In substituents SR¹, R¹ is preferably alkyl. Particularly preferably, SR¹ is thiomethyl.

In substituents SO₂R¹, R¹ is preferably H or alkyl. Particularly preferably, SO₂R¹ is methylsulfonyl.

In substituents OSO₂R¹, R¹ is hydrogen or alkyl. Particularly preferably, OSO₂R¹ is OSO₂CH₃ and OSO₂CF₃.

In substituents COR¹, R¹ is preferably H, alkyl or phenyl. Particularly preferably, COR¹ is formyl, acetyl or benzoyl.

In substituents CONR¹R², R¹ is preferably H or alkyl and R² is preferably H, alkyl or COR¹. Particularly preferably, CONR¹R² is CONH₂, CONHCH₃, CON(CH₃)₂ or CONHCOCH₃.

In substituents NR¹R², R¹ is preferably H, alkyl or phenyl-substituted alkyl and R² is H, alkyl or COR¹. Particularly preferably, NR¹R² is NH₂, NHCH₃, N(CH₃)₂, NH-benzyl or NHCOCH₃.

In substituents SO₂NR¹R², R¹ is preferably H or alkyl and R² is preferably H, alkyl or COR¹. Particularly preferably, SO₂NR¹R² is sulfamoyl.

In groups NR², R⁷ is preferably H, alkyl, phenyl-substituted alkyl or COR¹. Particularly preferably, NR² is NH, NCH₃, NCOCH₃ or NCH₂-phenyl.

In groups CONR², R² is preferably H, alkyl, phenyl-substituted alkyl or COR¹. Particularly preferably, CONR² is CONH, CONCH₃ or CONCH₂-phenyl.

In particular if L is phenyl or benzothienyl, this radical is bonded to D via an amide bond. In other words, D includes at least one group Z═CONR², whose carbonyl group is bonded to the phenyl or benzothienyl radical. In this case, R² is preferably hydrogen.

According to a particular embodiment of the present invention, compounds are used in which L is a radical of the formula

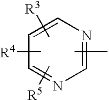

The preferred binding site of this radical L to D is the 2 position. Specific compounds of this embodiment are described in the examples of WO 96/02519, to which reference is hereby made.

According to a further particular embodiment of the present invention, compounds are used in which L is a radical of the formula

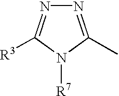

Specific compounds of this embodiment are mentioned in the examples of WO 96/02520, to which reference is hereby made.

According to a further particular embodiment of the present invention, compounds are used in which L is a radical of the formula

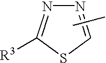

in which X is N or CR¹. For 1,3,4-thiadiazole groups, the binding to D preferably takes place via the 2 position and for 1,3-thiazole groups preferably via the 5 position. Specific compounds of this embodiment are mentioned in the examples of WO 96/02246, to which reference is made.

According to a further particular embodiment, compounds of the formula I are used in which L is one of the following radicals:

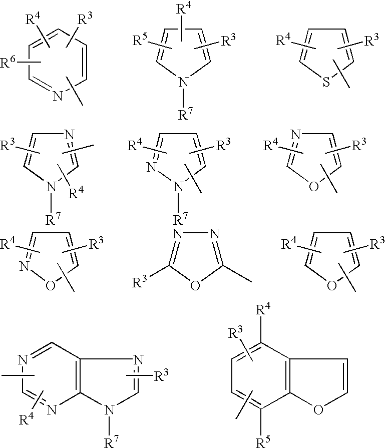

Particularly preferred radicals of these are pyridinyl and imidazolyl. Specific compounds of this embodiment are mentioned in the examples of WO 96/02246, to which reference is made.

According to a particular embodiment of the present invention, compounds are used in which B is a 7- or 8-membered saturated ring having one or two nitrogen heteroatoms, where the nitrogen heteroatoms are in the 1,4 or 1,5 position and the ring is bonded to the radical D in the 1 position and to the radical G in the 4 or 5 position and where the ring can moreover have a double bond in the 3 or 4 position. Accordingly, B is particularly preferably one of the following radicals:

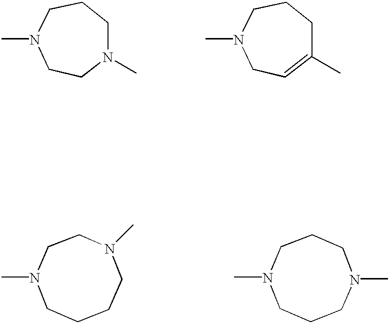

Specific compounds of this embodiment are mentioned in the examples of WO 97/25324, to which reference is made.

According to a particular embodiment of the present invention, compounds of the formula I are used in which L is a group of the formula

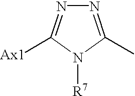

and B is one of the following radicals:

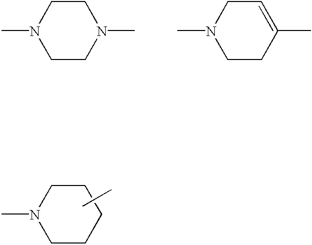

or if Ax1 is the 5- or 6-membered, heterocyclic aromatic ring, which can be substituted as indicated, B can also be one of the following radicals:

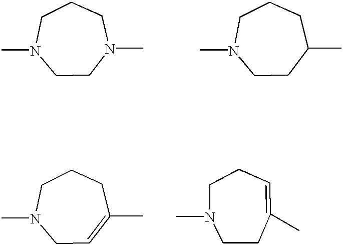

Specific compounds of this embodiment are mentioned in the examples of WO 99/02503, to which reference is made.

According to a further particular embodiment of the present invention, one of the compounds listed in the examples is used

According to a preferred embodiment of the present invention, compounds of the formula I are used in which

L is selected from

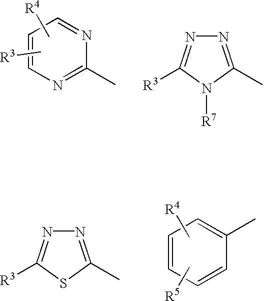

D is selected from -Z-C₃-C₆-alkylene or -Z-C₃-C₆-alkenylene, in particular -Z-CH₂CH═CHCH₂—, -Z-CH₂C(CH₃)═CHCH₂—, -Z-CH₂C(═CH₂)CH₂— or -Z-CH2CH(CH₃)CH₂— and especially -Z-CH₂CH₂CH₂— or -Z-CH₂CH₂CH₂CH₂—, linear -Z-C₇-C₁₀-alkylene radicals, in particular -Z-(CH₂)₇— or -Z-(CH₂)₈—, C₄-C₁₀-alkylene or C₄-C₁₀-alkenylene, in particular —(CH₂)₄—, —(CH₂)₅—, —(CH₂)₆—, —CH₂CH₂CH═CHCH₂—, —CH₂CH₂C(CH₃)═CHCH₂—, —CH₂C(═CH₂)CH₂— or —CH₂CH₂CH(CH₃)CH₂—;

B is selected from

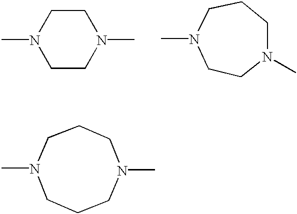

and

G is selected from optionally substituted phenyl or pyrimidinyl, where substituents independently of one another are preferably selected from alkyl, especially t-butyl or isopropyl, halogen, especially chlorine, CN, NO₂ and haloalkyl, especially CF₃ and CF₂H;

where Z is, of the meanings indicated above, in particular CONH if L is phenyl, and in the other cases is S, and R³, R⁴ and R⁵ are, of the meanings indicated above, independently of one another in particular hydrogen, amino, alkylamino or OH, R³ can moreover also be Ax1, where Ax1 is preferably phenyl, pyrrolyl or pyridinyl, and R⁷, of the meanings indicated above, is particularly hydrogen or preferably alkyl.

The compounds of the formula I described above can be prepared by

a) reacting a compound of the formula (II)

L-D-Y¹

in which Y¹ is a customary leaving group, such as halogen, alkanesulfonyloxy, arylsulfonyloxy etc., and Z has the abovementioned meanings,

with a compound of the formula (III)

H—B-G;

or

b) reacting a compound of the general formula (IV)

L-Di-Z¹H (IV)

in which Z¹ is O, NR¹ or S and D1 is C₁-C₁₀-alkylene or a bond, with a compound of the general formula V

yl-D₂-B-G (V)

where Y¹ has the meaning indicated above and D2 is C₂-C₁₀-alkylene, where D1 and D2 together have 1 to 18 C atoms;

or

c) reacting a compound of the general formula (VI)

L-Y¹ (VI)

in which Y¹ has the meaning indicated above, with a compound of the general formula VII

H-Z¹-D-B-G (VII)

in which Z¹ has the meanings indicated above; or

d) converting a compound of the formula (VIII)

NC-D-B-G (VIII)

into a compound of the type (IX)

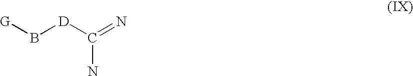

and reacting this in a known manner with a dicarbonyl compound; or

e) converting a compound of the general formula (X)

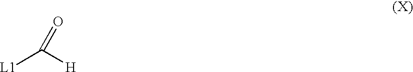

using reagents known from the literature, such as 1,3-propanedithiol, KCN/water, TMSCN (trimethylsilyl cyanide) or KCN/morpholine, as described, for example, in

Albright, Tetrahedron, 1983, 39, 3207 or

D. Seebach, Synthesis 1969, 17 and 1979, 19 or

H. Stetter, Angew. Chem. Int. Ed. 1976, 15, 639 or

van Niel et al., Tetrahedron 1989, 45, 7643

Martin et al., Synthesis 1979, 633,

to give the products (Xa) (by way of example with 1,3-propane-dithiol)

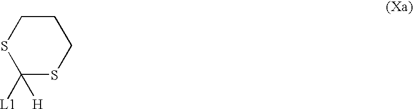

and then chain-lengthening with compounds of the general formula (XI)

yl-D₃-B-G (XI)

where Y¹ has the meaning indicated above and D3 is C₃-C₉-alkylene which can contain a group Z, where after deprotection or reduction compounds of the formula (Ia)

T-Z³-D2-B-G (Ia)

in which Z³ is CO or a methylene group and Z³ and D2 together have 4 to 10 C atoms, are obtained; or

g) reacting a compound of the formula (X) with a compound of the general formula (XII)

Y³-D-B-G (XII)

in which Y³ is a phosphorane or a phosphonic acid ester, analogously to customary methods, such as described in Houben weyl “Handbuch der Organischen Chemie” 4th Edition, Thieme Verlag Stuttgart, Volume V/1b pp. 383 ff or Vol. V/1c pp. 575 ff.

A process for the preparation of a compound of the formula I which includes the group COO or CONR⁷ can consist in reacting a compound of the general formula (XIII)

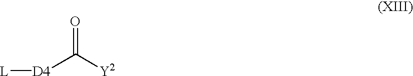

in which Y² is OH, OC₁-C₄-alkyl, Cl or, together with CO, is an activated ester group and D4 is C₀-C₈-alkylene, which can contain a group Z, with a compound of the formula (XIV)

Z²-D-B-G (XIV)

in which Z² is OH or NR⁷, where L, D, B and G have the meanings indicated above.

The compounds of the formula III are starting compounds for the preparation of compounds of the formulae V, VII and VIII.

Compounds of the formula III are prepared by standard methods, such as described in J. A. Kiristy et al., J. Med. Chem. 1978, 21, 1303 or C. B. Pollard, J. Am. Chem. Soc. 1934, 56, 2199, or by

a) reacting a compound of the general formula (XV)

HB3 (XV)

in which B3 is

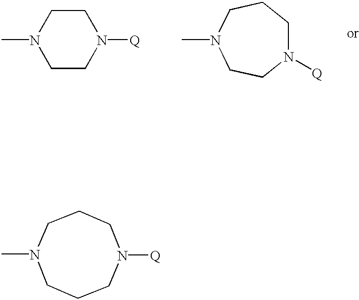

and Q is H or a customary amino protective group, e.g. butyloxycarbonyl, benzyl, or methyl, in a known manner with a compound of the general formula (XVI)

Y⁴-G (XVI)

in which Y⁴ is B(OH)₂, —SnBu₃, trifluoromethanesulfonyloxy or has the meanings indicated for Y¹; or

b) reacting a compound of the general formula (XVII)

Q-B4 (XVII)

in which B4 is

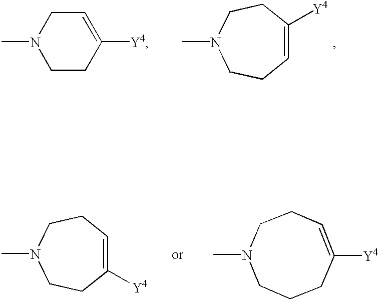

and Y4 and Q are have the meanings indicated above

with a compound of the general formula (XVIII)

Y⁵-G (XVIII)

in which Y⁵ is boron derivatives, such as B(OH)₂ or a metal-containing leaving group, e.g. SnR³ (R=butyl or phenyl) or zinc halide, if Y⁴ is halogen or trifluoromethylsulfonyloxy; or in which Y⁵ is halogen or trifluoromethylsulfonyloxy if Y⁴ is boron derivatives, such as B(OH)₂ or a metal-containing leaving group, e.g. SnR³ (R=butyl or phenyl) or zinc halide, according to known processes, such as described in

S. Buchwald et al., Angew. Chem. 1995, 107, 1456 or

J. F. Hartweg et al., Tetrahedron Lett 1995, 36, 3604 and

J. K. Stille et al., Angew. Chem. 1986, 98, 504 or

Pereyre M. et al., “Tin in Organic Synthesis”, Butterworth 1987; or

c) reacting a compound of the general formula (XIX)

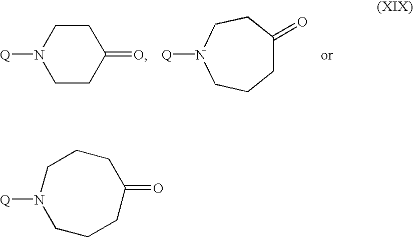

with a compound M-G,

in which M is a metal such as Li, MgY⁶ and Y⁶ is bromine, chlorine or iodine.

M-G can be obtained by methods known from the literature.

Compounds of type B are either known or they can be prepared analogously to known processes, such as

1,4- and 1,5-diazacycloalkanes:

L. Börjeson et al. Acta Chem. Scand. 1991, 45, 621

Majahrzah et al. Acta Pol. Pharm. 1975, 32, 145

1,4-diazacyclooct-6-enes:

W. Schroth et al. Z. Chem. 1969, 9, 143

1-azacyclooctanones:

N. J. Leonard et al. J. Org. Chem. 1964, 34, 1066

1-azacycloheptanones:

A. Yokoo et al. Bull Chem. Soc. Jpn. 1956, 29, 631

Compounds of type L and G are either known or can be prepared by known processes such as described in A. R. Katritzky, C. W. Rees (ed.) “Comprehensive Heterocyclic Chemistry”, Pergamon Press, or “The Chemistry of Heterocyclic Compounds”, J. Wiley & Sons Inc. NY and the literature cited there or the patent literature cited above.

The compounds of the type of formula (IV) are either known or can be prepared by known processes, such as are described in A. R. Katritzky, C. W. Rees (ed.) “Comprehensive Heterocyclic Chemistry”, Pergamon Press, or “The Chemistry of Heterocyclic Compounds” J. Wiley & Sons Inc. NY and the literature cited there or in S. Kubota et al. Chem. Pharm. Bull. 1975, 23, 955 or Vosilevskii et al. Izv. Akad. Nauk. SSSR Ser. Khim. 1975, 23, 955.

Further synthesis possibilities for the person skilled in the art result from the process outlined in WO 96/02519, WO 96/02520, WO 96/02249, WO 96/02246, WO 97/25324, WO 99/02503 and WO 98/05178 and in particular from the illustrative preparation examples indicated there.

Of the abovementioned compounds, according to the invention those are particularly advantageous which have a high affinity for dopamine D₃ receptors. In this sense, particularly preferred compounds are those which in vitro have Kᵢ values of less than 1 μM and especially of less than 200 nM. Suitable test procedures for the selection of these compounds are known to the person skilled in the art. For example, binding affinities for D₃ receptors can be determined in receptor binding studies by means of the displacement of [¹²⁵I]-iodosulpiride.

Particularly of advantage are those of the previously described compounds which bind selectively to dopamine D₃ receptors. Of importance in this connection are especially selectivities to D₁ receptors, D₄ receptors, α1 and/or α2 adrenergic receptors, serotinergic receptors, especially 5HT1A and 5HT7, muscarinergic receptors, histamine receptors, opiate receptors and in particular to dopamine D₂ receptors.

Relevant test procedures for the determination of binding affinities for these receptors are also known to the person skilled in the art. Receptor binding studies on D₁, D₂ and D₄ receptors can be carried out, for example, by means of the displacement of [³H)SCH23390, (¹²⁵I]iodosulpiride or [³H]spiperone.

Of particular importance according to the invention is the selectivity Kᵢ(D₂)/Kᵢ(D₃), which is preferably at least 10, better still at least 50 and particularly advantageously at least 100.

With respect to the effector function, according to a particular embodiment of the present invention those of the abovementioned compounds which are partial dopamine D₃ receptor agonists are used.

Adequately known test procedures are also available to the person skilled in the art for the determination of effector functions. For example, D₃ receptor agonists lead in D₃-expressing cells to a decrease in intracellular cAMP levels, which can be determined directly via the determination of cAMP, which is known per se, or indirectly, for example, by means of cAMP-dependent reporters. A further example is the increase in the affinity of the α-subunit of G proteins for GTP, which can be measured via the stimulation of the binding of ³⁵S-GTP to G proteins. The stimulation of ³H-thymidine incorporation into D₃ receptor-expressing neuroblastoma cells by D₃ receptor agonists and the change in intracellular pH is due to changes in acid excretion are further possibilities of assessing effector functions of compounds to be used according to the invention.

Suitable models in the field of addictive disorders are based on behavior patterns, which are typical of addiction, of animals to which psychotropic substances are administered. For example, animals are trained to press a button on treatment with active compound and another button on treatment with placebo. The test substance is investigated for its ability to induce a behavior of this type, to suppress the response induced by a further psychotropic substance or to replace a further psychotropis substance. In a similar manner, the animals can be trained to the effect that they prefer a certain place. A further example is based on the ability of an active compound to cause an animal to administer this active compound to itself, usually by activating a pump which is attached to a catheter. The ability of an active compound to produce physical symptoms in the case of withdrawal following a chronic administration also represents a possibility of assessing the psychotropic potential or the ability to counteract the psychotropic action of a specific active compound.

Those compounds are very particularly advantageous which have both the advantageous binding properties to dopamine D₃ receptors described above, and exert one or more of the effector functions described by way of example. Preferably, such compounds in D₂-expressing cells do not lead or only lead in significantly higher concentration to the effects described above.

Compounds are advantageous which themselves have no psychotropic action. This can also be observed in the test on rats which, after administration of compounds which can be used according to the invention, cut down the self-administration of psychotropic substances, for example cocaine.

The test systems described above and further test systems which are similarly suitable can form the basis for in vitro screening procedures, preferably for primary screening, with which, from the compounds described, those can be picked out which offer particular advantages with respect to the use according to the invention. This is automatable. Screening robots are used for the efficient evaluation of the individual assays, which are preferably arranged on microtiter plates.

A particularly effective technology for carrying out procedures of this type is the scintillation proximity assay, called SPA for short, known in the field of active compound screening. Kits and components for carrying out this assay can be obtained commercially, for example from Amersham Pharmacia Biotech.

A further particularly effective technology for carrying out procedures of this type is the FlashPlate technology known in the field of active compound screening. Kits and components for carrying out this assay can be obtained commercially, for example from NEN Life Science Products. This principle is likewise based on microtiter plates (96-well or 384-well), which are coated with scintillation substance.

Further test procedures especially suitable for secondary screening are based on in-vitro and in-vivo models for indications to be treated according to the invention.

In the context of treatment, the use according to the invention of the compounds described comprises a process. In this process, an efficacious amount of one or more compounds, as a rule formulated according to pharmaceutical or veterinary medicinal practice, is administered to the individual to be treated, preferably a mammal, in particular a human, agricultural or domestic animal. Whether such a treatment is indicated and in what form it has to be carried out depends on the individual case and is subject to a medical assessment (diagnosis) to develop the present signs, symptoms and/or malfunctions, risks of developing certain signs, symptoms and/or malfunctions, and includes further factors.

As a rule, the treatment is carried out by administration one or more times per day together or alternately with other active compounds or active compound-containing preparations, such that an individual to be treated is administered a daily dose of approximately 1 to 1000 mg/kg of body weight in the case of oral administration, preferably of approximately 0.1 to 100 mg/kg of body weight in the case of parenteral administration.

The invention also relates to the production of pharmaceutical compositions for the treatment of an individual, preferably of a mammal, in particular of a human, agricultural or domestic animal. Thus the ligands are usually administered in the form of pharmaceutical compositions which comprise a pharmaceutically tolerable excipient with at least one ligand according to the invention and, if appropriate, further active compounds. These compositions can be administered, for example, by the oral, rectal, transdermal, subcutaneous, intravenous, intramuscular or intranasal route.

Examples of suitable pharmaceutical formulations are solid pharmaceutical forms, such as powders, granules, tablets, in particular film-coated tablets, pastilles, sachets, cachets, sugar-coated tablets, capsules such as hard and soft gelatin capsules, suppositories or vaginal pharmaceutical forms, semisolid pharmaceutical forms, such as ointments, creams, hydrogels, pastes or patches, as well as liquid pharmaceutical forms, such as solutions, emulsions, in particular oil-in-water emulsions, suspensions, for example lotions, injection and infusion preparations, eye and ear drops. Implanted delivery devices can also be used for the administration of inhibitors according to the invention. In addition, liposomes or microspheres can also be used.

In the production of the compositions, inhibitors according to the invention are usually mixed or diluted with an excipient. Excipients can be solid, semisolid or liquid materials which serve as a vehicle, carrier or medium for the active compound.

Suitable excipients are listed in the relevant pharmaceutical monographs. In addition, the formulations can comprise pharmaceutically acceptable carriers or customary excipients, such as lubricants; wetting agents; emulsifying and suspending agents; preservatives; antioxidants; antiirritant substances; chelating agents; coating excipients; emulsion stabilizers; film formers; gel formers; odor-masking agents; flavor corrigents; resins; hydrocolloids; solvents; solubilizers; neutralizing agents; permeation accelerators; pigments; quaternary ammonium compounds; refatting and superfatting agents; ointment, cream or oil bases; silicone derivatives; spreading excipients; stabilizers; sterilizing agents; suppository bases; tablet excipients, such as binders, fillers, lubricants, disintegrants or coatings; propellants; drying agents; opacifying agents; thickening agents; waxes; plasticizers; white oils. An embodiment in this respect is based on expert knowledge, such as is presented in Fiedler, H. P., Lexikon der Hilfsstoffe für Pharmazie, Kosmetik und angrenzende Gebiete [Encyclopedia of Excipients for Pharmacy, Cosmetics and related areas], 4th Edition, Aulendorf: ECV-Editio-Kantor-Verlag, 1996.

The present invention is illustrated in greater detail with the aid of the following examples, without being restricted thereto.

### EXAMPLE 1

5-((3-[4-(3,5-Dichlorophenyl)-1-piperazinyl]propyl}sulfanyl)-1,3,4-thiadiazole-2-amine;

### EXAMPLE 2

5-[(2-Methyl-3-(4-[3-(trifluoromethyl)phenyl]-1-piperazinyl)propyl)sulfanyl]-1,3,4-thiadiazole-2-amine;

### EXAMPLE 3

2-[{3-(4-[3-(Trifluoromethyl)phenyl]-1-piperazinyl}propyl)sulfanyl]-4-pyrimidinole;

### EXAMPLE 4

5-(6-(4-(3-(Trifluoromethyl)phenyl]-1-piperazinyl)hexyl)-1,3,4-thiadiazole-2-amine;

### EXAMPLE 5

1-{(2E)-4-[(4-Hydroxy-2-pyrimidinyl)sulfanyl]-2-butenyl}-4-[3-(trifluoromethyl)phenyl]piperazineium dichloride;

### EXAMPLE 6

4-Methyl-5-[(3-(4-[3-(trifluoromethyl)phenyl]-1-piperazinyl)propyl)sulfanyl]-4H-1,2,4-triazole-3-amine;

### EXAMPLE 7

2-([3-(4-[3-(Trifluoromethyl)phenyl]-3,6-dihydro-1(2H)-pyridinyl)propyl]sulfanyl)-4-pyrimidinole;

### EXAMPLE 8

2-({3-[4-(3-Isopropylphenyl)-1-piperazinyl)propyl}sulfanyl)-4-pyrimidinole;

### EXAMPLE 9

5-[(3-{4-[6-(Trifluoromethyl)-2-pyridinyl]-1-piperazinyl}propyl)sulfanyl}-1,3,4-thiadiazole-2-amine;

### EXAMPLE 10

5-[((2E)-4-(4-[6-(Trifluoromethyl)-2-pyridinyl]-1-piperazinyl}-2-butenyl)sulfanyl]-1,3,4-thiadiazole-2-amine;

### EXAMPLE 11

5-({3-(4-(3,5-Di-tert-butylphenyl)-1-piperazinyl]propyl}sulfanyl)-4-methyl-4H-1,2,4-triazole-3-amine;

### EXAMPLE 12

5-({7-[4-(3-Isopropylphenyl)-1-piperazinyl]heptyl}sulfanyl)-1,3,4-thiadiazole-2-amine;

### EXAMPLE 13

4-Ethyl-5-[(3-(4-[3-(trifluoromethyl)phenyl)-1-piperazinyl)propyl)sulfanyl]-4H-1,2,4-triazole-3-amine;

### EXAMPLE 14

2-[(3-{4-[2-Tert-butyl-6-(trifluoromethyl)-4-pyrimidinyl)-1-piperazinyl}propyl)sulfanyl]-4-pyrimidinole, in particular its fumaric acid salt;

### EXAMPLE 15

3-(4-{8-[(5-Amino-1,3,4-thiadiazol-2-yl)sulfanyl]octyl)-1-piperazinyl)benzonitrile;

### EXAMPLE 16

5-[(3-{4-[6-Methyl-2-(1H-pyrrol-1-yl)-4-pyrimidinyl]-1-piperazinyl}propyl)sulfanyl]-1,3,4-thiadiazole-2-amine;

### EXAMPLE 17

4-Methyl-5-[(3-{4-(6-methyl-2-(1H-pyrrol-1-yl)-4-pyrimidinyl]-1-piperazinyl}propyl)sulfanyl]-4H-1,2,4-triazole-3-amine;

### EXAMPLE 18

5-[{(2E)-4-(4-[2-Tert-butyl-6-(trifluoromethyl)-4-pyrimidinyl]-1-piperazinyl)-2-methyl-2-butenyl}sulfanyl]-4-methyl-4H-,2,4-triazole-3-amine;

### EXAMPLE 19

1-[3-(Difluoromethyl)phenyl)-4-(3-([4-methyl-5-(methylammonio)-4H-1,2,4-triazol-3-yl]sulfanyl)propyl)piperazin-4-ium dichloride;

### EXAMPLE 20

5-({3-[4-(2,6-Di-tert-butyl-4-pyrimidinyl)-1-piperazinyl]propyl}sulfanyl)-1,3,4-thiadiazole-2-amine;

The compounds listed in Examples 1 to 20 are selective dopamine D₃ ligands whose affinity for D₂ receptors or D₃ receptors was determined for a ratio Kᵢ(D₂)/Kᵢ(D₃) of more than 10 according to the methods indicated in the reference examples.

### Reference Examples

Biological Investigations—Receptor Binding Studies

1) D₃ Binding Test

For the binding studies, cloned human D₃ receptor-expressing CCL 1,3 mouse fibroblasts, obtainable from Res. Biochemicals Internat. One Strathmore Rd., Natick, Mass. 01760-2418 USA, were employed.

Cell Preparation

The D₃-expressing cells were proliferated in RPMI-1640 using 10% fetal calf serum (GIBCO No. 041-32400 N); 100 U/ml of penicillin and 0.2% of streptomycin (GIBCO BRL, Gaithersburg, Md., USA). After 48 h, the cells were washed with PBS and incubated for 5 min with 0.05% trypsin-containing PBS. Neutralization with medium was then carried out and the cells were collected by centrifugation at 300 g. For the lysis of the cells, the pellet was briefly washed with lysis buffer (5mM tris-HCl, pH 7.4 containing 10% glycerol) and then incubated at 4° C. for 30 min in a concentration of 10⁷ cell/ml of lysis buffer. The cells were centrifuged at 200 g for 10 min and the pellet was stored in liquid nitrogen.

Binding Tests

For the D₃ receptor binding test, the membranes were suspended in incubation buffer (50 mM tris-HCl, pH 7.4 containing 120 m NaCl, 5 mM KCl, 2 mM CaCl₂, 2 mM MgCl₂, 10 μM quinolinol, 0.1% of ascorbic acid and 0.1% of BSA) in a concentration of about 10⁶ cell/250 μl test batch and incubated at 30° C. with 0.1 nM ¹²⁵iodosulpiride in the presence and absence of test substance. The nonspecific binding was determined using 10⁻⁶M spiperone.

After 60 min, the free and the bound radio ligands were separated on a Skatron cell harvester (Skatron, Lier, Norway) by means of filtration through GF/B glass fiber filters (Whatman, England) and the filters were washed with ice-cold tris-HCl buffer, pH 7.4. The radioactivity collected on the filters was quantified using a Packard 2200 CA liquid scintillation counter.

The determination of the Kᵢ values was carried out by means of nonlinear regression analysis using the LIGAND program.

2) D₂ Binding Test

Cell Culture

HEK-293 cells having stably expressed human dopamine D2A receptors were cultured using Glutamax I™ and 25 mM HEPES containing 10% fetal calf serum albumin in RPMI 1640. All media contained 100 units per ml of penicillin and 100 μg/ml of streptomycin. The cells were kept at 37° C. in a moist atmosphere containing 5% CO₂.

The cell preparation for binding studies was carried out by trypsinization (0.05% trypsin solution) for 3-5 minutes at room temperature. The cells were then centrifuged at 250 g for 10 minutes and treated at 4° C. with lysis buffer (5 mM tris-HCl, 10% glycerol, pH 7.4) for 30 minutes. After centrifugation at 250 g for 10 minutes, the residue was stored at −20° C. until use.

Receptor Bindings Tests

Dopamine D₂ receptor “low affinity state” containing ¹²⁵I-spiperone (81 TBq/mmol, Du Pont de Nemours, Dreieich)

The batches (1 ml) were composed of 1×10⁵ cells in incubation buffer (50 mM tris, 120 mM NaCl, 5 mM KCl, 2 mM MgCl₂ and 2 mM CaCl₂, pH 7.4 using HCl) and 0.1 mM ¹²⁵I-spiperone (total binding) or additionally 1 μM haloperidol (nonspecific binding) or test substance.

After incubation had taken place at 25° C. for 60 minutes, the batches were filtered through GF/B glass fiber filters (Whatman, England) on a Skatron cell harvester (Zinsser, Frankfurt) and the filters were washed with ice-cold 50 mM tris-HCl buffer, pH 7.4. The radioactivity collected on the filters was quantified using a Packard 2200 CA liquid scintillation counter.

The assessment was carried out as in a).

The determination of the Kᵢ values was carried out by means of nonlinear regression analysis using the LIGAND program or by conversion of the IC₅₀ values with the aid of the formula of Cheng and Prusoff.

In these tests, the compounds according to the invention show very good affinities for the D₃ receptor (<1 μmolar, in particular<100 nmolar) and high selectivities for the D₂ receptor.

1. The use of at least one compound of the general formula I
L-D-B-G (I)
in which
L is a 5- or 6-membered aromatic heteromonocyclic system L1 having 1, 2 or 3 heteroatoms selected independently of one another from O, N and S
or an aromatic or heteroaromatic radical selected from the group L2 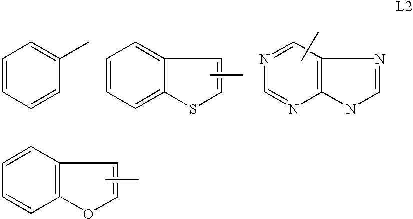
where L optionally has 1, 2, 3 or 4 substituents which independently of one another are selected from Cₗ-C₈-alkyl which is optionally substituted by OH, OC₁-C₈-alkyl, phenyl or halogen; OR¹, C₂-C₆-alkenyl, C₂-C₆-alkynyl, C₃-C₈-cycloalkyl, halogen, CN, CONR¹R², COOR¹, NO₂, NR¹R², SR¹, SO₂R¹, SO₂NR¹R², OSO₂R¹, Ax1 or phenoxy which is optionally substituted by C₁-C₆-alkyl, OC₁-C₆-alkyl or halogen; C₁-C₆-alkanoyl or benzoyl;
in which
Ax1 is phenyl, naphthyl or a 5- or 6-membered heterocyclic aromatic ring having 1, 2, 3 or 4 heteroatoms,
where Ax1 optionally has 1, 2, 3 or 4 substituents which independently of one another are selected from C₁-C₆-alkyl which is optionally substituted by OH, OC₁-C₆-alkyl, halogen or phenyl; C₁-C₆-alkoxy, C₂-C₆-alkenyl, C₂-C₆-alkynyl, C₃-C₆-cycloalkyl, halogen, CN, COR¹, COOR¹, NR¹R², NO₂, SR¹, SO₂R¹, SO₂NR¹R², or phenyl which is optionally substituted by C₁-C₆-alkyl, OC₁-C₆-alkyl, NR¹R², CN, CF₃, CHF₂, or halogen; and where the heterocyclic aromatic ring mentioned can optionally be fused to a phenyl ring;
R¹ is H, C₃-C₆-cycloalkyl, phenyl or C₁-C₈-alkyl which is optionally substituted by OH, OC₁-C₈-alkyl, halogen or phenyl;
R² has the meanings indicated for R¹ or is COR¹ or CO₂R¹;
D is a C₁-C₁₈-alkylene group or a C₁-C₁₈-alkylene group which includes at least one group Z which is selected from O, S, NR², C₃-C₆-cycloalkyl, CO, CONR², CH₂, a double and a triple bond, where R² is as defined above;
B is a 6-, 7- or 8-membered saturated ring having one or two nitrogen heteroatoms, where the nitrogen heteroatoms are located in the 1,4 or 1,5 position and the ring is bonded to the radical D in the 1 position and to the radical G in the 4 or 5 position and where the ring can moreover have a double bond in the 3 or 4 position;
G is phenyl, pyridyl, pyrimidinyl or triazinyl,
where G can optionally have 1, 2, 3 or 4 substituents which independently of one another are selected from OR¹, C₁-C₈-alkyl, C₂-C₈-alkenyl, C₂-C₈-alkynyl, C₁-C₆-alkoxy-C₁-C₆-alkyl, halogen-C₁-C₆-alkyl, halogen-C₁-C₆-alkoxy, halogen, CN, CO₂R¹, NO₂ SO₂R¹, SO₃R¹, NR¹R², SO₂NR¹R², SR¹, a 5- or 6-membered carbocyclic, aromatic or nonaromatic ring and a 5- or 6-membered heterocyclic aromatic or nonaromatic ring having 1 or 2 heteroatoms independently of one another selected from O, S and N, where the carbocyclic or the heterocyclic ring is optionally substituted by C₁-C₈-alkyl, phenyl, phenoxy, halogen, OC₁-C₈-alkyl, OH, NO₂, or CF₃,
where G can optionally be fused to a carbocyclic or heterocyclic ring of the type defined above;
and their salts with physiologically tolerable acids,
for the production of a medicament for treating disorders of the central nervous system which respond to dopamine D₃ ligands.

2. The use as claimed in claim 1 for the treatment of psychiatric disorders.

3. The use as claimed in claim 2 for the treatment of addiction.

4. The use as claimed in claim 3 for the treatment of disorders which are mediated by psychotropic substances, in particular by opioids or cocaine.

5. The use as claimed in one of claims 1 to 4 of at least one compound of the formula I, in which L is a group of the formula 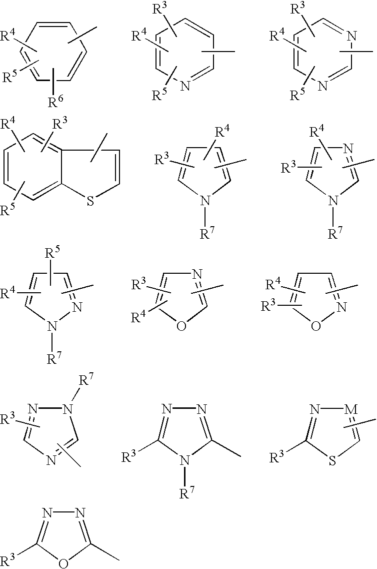
in which
R³ is Ax1, OR¹, R¹, C₂-C₆-alkenyl, C₂-C₆-alkynyl, halogen, CN, CONR¹R², COOP¹, NO₂, NR¹R², SR¹, OSO₂R¹, SO₂R¹;
R⁴ to R⁶ independently of one another are H, C₁-C₆-alkyl, OR¹, CN, NR¹R², SR¹, CF₃;
R⁷ is H, C₁-C₆-alkyl or C₃-C₆-cycloalkyl;
M is N or CR¹;
and the other radicals have the meaning indicated in claim 1.

6. The use as claimed in one of claims 1 to 4 of at least one compound of the formula I, in which L is 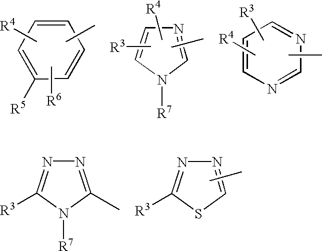
in which
R³ is AX1, R¹, COOR¹, NO₂, NR¹R², SR¹, OSO₂CF₃, SO₂R¹, CF₃, CHF₂,
R⁴ to R⁶ are H, C₁-C₆-alkyl, OR¹, NR²R²;
R⁷ is H, C₁-C₆-alkyl;
and the other radicals have the meaning indicated in claim 1.

7. The use as claimed in one of claims 1 to 4 of at least one compound of the formula I, in which Ax1 is a substituent of the formula 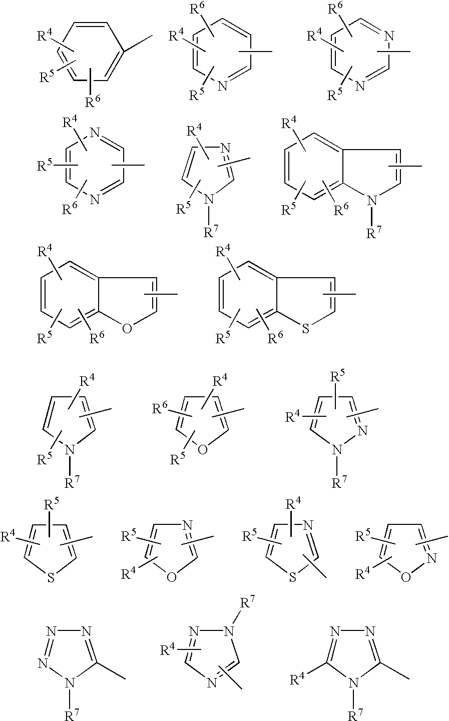
in which
R⁴ to R⁶ have the meanings indicated in claim 5 or 6;
R⁷ is C₁-C₄-alkyl;
and the other radicals have the meanings indicated in claim 1, 5 or 6.

8. The use as claimed in one of claims 1 to 4 of at least one compound of the formula I, in which Ax1 is a substituent of the formula 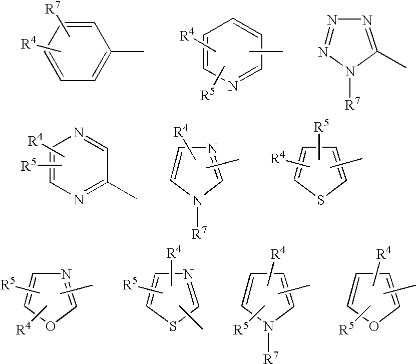
in which the other radicals have the meanings indicated in claims 1 or 5 to 7.

9. The use as claimed in one of claims 1 to 4 of at least one compound of the formula I, in which B is a group of the formula 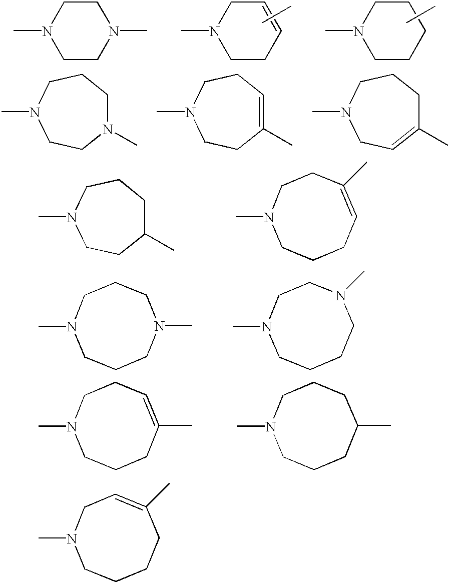
and the other radicals have the meanings indicated in claims 1 or 5 to 8.

10. The use as claimed in one of claims 1 to 4 of at least one compound of the formula I, in which B is a group of the formula 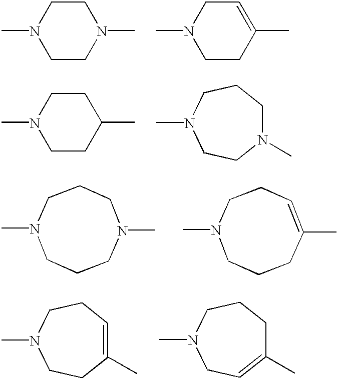
and the other radicals have the meanings indicated in claims 1 or 5 to 8.

11. The use as claimed in one of claims 1 to 4 of at least one compound of the formula I, in which B is a group of the formula 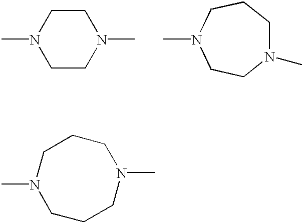
and the other radicals have the meanings indicated in claims 1 or 5 to 8.

12. The use as claimed in one of claims 1 to 4 of at least one compound of the formula I, in which D is a CONR²—C₃-C₁₀-alkylene group when L is a group L2, or, when L is a group L1, D is an alkylene group or an alkylene group comprising Z having 4 to 10 or 3 to 10 carbon atoms respectively, and the other radicals have the meanings mentioned in claims 1 or 5 to 11.

13. The use as claimed in one of claims 1 to 4 of at least one compound of the formula I
L-D-B-G (I)
in which D is -Z-C₃-C₆-alkylene, in particular -Z-CH₂CH₂CH₂—, -Z-CH₂CH₂CH₂CH₂—, -Z-CH₂CH═CHCH₂—, -Z-CH₂C(CH₃)═CHCH₂—, -Z-CH₂C(═CH₂)CH₂—, -Z-CH₂CH(CH₃)CH₂— or is a linear -Z-C₇-C₁₀-alkylene radical;
and the other radicals have the meanings indicated in claims 1 or 5 to 11.

14. The use as claimed in one of claims 1 to 4 of at least one compound of the formula I, in which Z is CH₂, O or S, and the other radicals have the meanings indicated in claims 1 or 5 to 13.

15. The use as claimed in one of claims 1 to 4 of at least one compound of the formula I, in which G is optionally substituted phenyl, 2-pyridinyl, 3-pyridinyl, 4-pyridinyl, 2-pyrimidinyl, 4(6)-pyrimidinyl or 5-pyrimidinyl, and the other radicals have the meanings mentioned in claims 1 or 5 to 14.

16. The use as claimed in one of claims 1 to 4 of at least one compound of the formula I, in which G has one or two substituents in the m and/or p position, which independently of one another are selected from C₁-C₆-alkyl, CF₃, CHF₂, NO₂, CN, CO₂R¹, halogen, phenyl, pyrrolyl, imidazolyl, pyrazolyl, thienyl, cyclopentyl and cyclohexyl;
and the other radicals have the meanings indicated in claims 1 or 5 to 15.

17. The use as claimed in one of claims 1 to 4 of a compound of the formula I, in which L is selected from 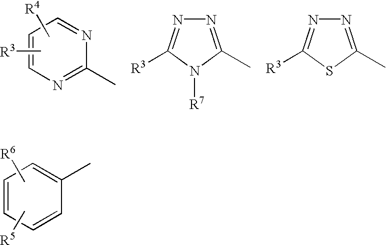
D is -Z-(CH₂)₃— or -Z-(CH₂)₄,
B is 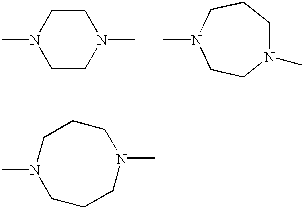
and
G is optionally substituted phenyl,
and the other radicals have the meanings mentioned in claims 1 or 5 to 16.
